(19) United States
(12) Patent Application Publication
(10) Pub. No.: US 2024/0334883 A1
(43) Pub. Date: Oct. 10, 2024

(54) METHOD FOR ANTIFUNGAL TREATMENT OF TURF

(71) Applicant: TEAM GREEN LIGHT, Fareins (FR)

(72) Inventor: Cédric MARTINOTTI, Fareins (FR)

(73) Assignee: TEAM GREEN LIGHT, Fareins (FR)

(21) Appl. No.: 18/293,524

(22) PCT Filed: Jul. 29, 2022
(86) PCT No.: PCT/FR2022/051532

§ 371 Date: Jan. 30, 2024

(30) Foreign Application Priority Data
Jul. 30, 2021 (FR) 21/08325

(51) Int. Cl. A01G 7/04; A01G 20/30; A61L 2/00; A61L 2/26

(52) U.S. Cl. CPC A01G 7/045; A01G 20/30; A61L 2/0052; A61L 2/26; A61L 2202/11

(57) ABSTRACT

A method for the antifungal treatment of a turf includes a step of applying blue light on said turf.

The present invention relates to a method for the antifungal treatment of a turf, comprising a step of applying blue light on said turf.

Some microscopic fungi (whose size is smaller than 1 mm) or so-called “phytopathogenic” micromycetes could be the cause of diseases of turf, particularly in wet and shaded places. These diseases of fungal origin (or fungal diseases) are particularly problematic during prolonged periods of rain or of high humidity, and could damage the turf on the long run, for example in parks, gardens and sports fields.

Currently, fungicidal treatments are primarily used to fight these fungal phytopathologies. For example, the strains of fungi at the origin of cold fusarium wilt which could affect the golf courses are sensitive to several different families of fungicides (triazoles, strobilurines, phenylpyrolles or dicarboximides). However, these fungicidal products result in resistance phenomena, by destroying the good (for example, commensal fungi) as well as the bad (for example, saprophytic fungi or parasitic fungi) deep in the soil. Furthermore, these treatments are potentially ecotoxic and will probably be prohibited in a near future.

It is also described that the exposure of a microorganism to a light radiation alters its integrity and even leads to removal thereof. As example, the document WO 2020/084199 discloses the treatment of biological surfaces, i.e. portions of the body of a human or of an animal, by application of an electromagnetic radiation in the form of light of two different energy levels.

Alternatively, another solution consists in proceeding with a treatment of the turf through the application of a C-type ultraviolet radiation (or UV-C) which has a short wavelength (280-100 nm) and which is the most energetic UV radiation but the most harmful. It is known that the maximum absorbance by DNA is about 250 nm. Consequently, the application of an UV-C radiation allows denaturing/breaking the DNA of the microorganisms included in the turf such as viruses or fungi which are at the origin of fungal diseases. However, this UV-C radiation acts by destroying the good (auxiliary microorganisms exerting beneficial effects) as well as the bad (pathogenic and/or parasitic microorganisms). As example, this treatment method induces the elimination of endophytes which secrete alkaloids which are toxic to some insects and have a repellent effect on others. Furthermore, this type of UV radiation may prove dangerous to humans due to its DNA denaturation action.

The document U.S. Pat. No. 9,131,645 describes a method for improving the characteristics of plants by exposure thereof to high-intensity illumination. In turn, the document JP2016010396 discloses a method using different colors of visible light, with an application during the night on some types of leaved plants (in particular, strawberry), so as to induce phenol production by these plants and to make them resistant to fusarium wilt.

Yet, it has now been discovered that, quite surprisingly, blue light allows treating the fungal diseases of turf, by causing the fungi to regression but without eradicating them completely, so that no resistance phenomenon occurs. Blue light is not hazardous to humans, and is not toxic to turf. Quite the contrary, this blue light allows even reinforcing the tilling and rooting of the plants of the turf, which allows recolonizing the areas that have been damaged by the fungi.

Thus, the present invention relates to a method for the antifungal treatment of a turf, comprising a step of applying blue light on said turf.

The method according to the present invention allows not only completing or replacing the phytosanitary treatments, also containing and/or causing the fungal infection to regression by limiting the sporulation and the development of the mycelium, and reinforcing the plant in particular in order not to create a void and/or a resistance of the microscopic fungus (fungi) to the treatment according to the invention.

In the context of the present invention:
By “antifungal treatment method”, it should be understood any method allowing acting directly on microscopic fungi (or micromycetes, whose size is smaller than 1 mm), the mycelium and/or the spores, considered as phytopathogens and present in the turf, by maintaining/controlling development thereof and/or activity thereof (for example the production and secretion of mycotoxins), by causing regression thereof, by stopping development thereof and their mode of reproduction, so as to weaken them without killing them, or stressing them. In fine, said antifungal treatment method allows maintaining or reducing the pathogenic effect(s) and therefore the negative impact of the micromycetes, of the mycelium and/or of the spores on turf, while reinforcing the plant, in particular the vigor of the plant;
By “turf” (also called artificial lawn, or more commonly lawn), it should be understood any endemic or sown surface comprising a high density of fine herbs selected, essentially from among grasses such as agrostises (for example, Agrostis stolonifera, Agrostis capillaris, Agrostis castellana, Agrostis canina), meadow-grass (for example, smooth meadow-grass, common meadow-grass, annual meadow-grass), fescue (for example, red fescue, high fescue, hard ovine fescue) and/or English ryegrass. For example, it may consist of ornamental or aggregate turf in parks and gardens, or sports turf (golf, tennis, football, hockey, hippodrome, etc.); and
By “blue light”, it should be understood the part of the spectrum of the visible light having one (or more) wavelength(s) comprised between 380 and 500 nm.

### FIGURES

FIG. 1: Photograph representing the turf of a golf course composed of agrostis and of meadow-grasses and suffering from cold fusarium wilt (F. nivale, M. nivale and M. majus; arrows) before treatment according to the invention (photograph to the left) and after daytime and nighttime treatment by applying blue light at a wavelength of 450 nm (photograph to the right) at a rate of 21 applications for 30 days with a frequency of 2 applications every day for 4 days, then 1 application every 2 days.

FIG. 2: Photograph representing the resumption of the growth of the grasses (area delimited by the dotted line) in the turf of a golf course composed of agrostis and of meadow-grasses, infected with cold fusarium wilt (Fusarium nivale, Microdochium nivale and Microdochium majus) and then treated in daytime and in nighttime by applying blue light at a wavelength of 450 nm, at a rate of 21 applications for 30 days with a frequency of 2 applications every day for 4 days, then 1 application every 2 days.

FIG. 3: Photograph representing the turf of a golf course composed of agrostis and annual meadow-grass, infected with cold fusarium wilt (M. nivale; arrows) and fairy rings (Marasmius oreade; “green” ring identified between the dotted lines) before treatment according to the invention (photograph to the left) and after treatment in daytime by application of blue light at a wavelength of 450 nm (photograph to the right) at a rate of 10 applications for 30 days with a frequency of 1 application every 3 days.

FIG. 4: photographs representing the in vitro culture of M. nivale 2, 3, 4, 6 and 12 days after being cultured without processing (series of photographs to the left) and with a treatment by application of blue light at a wavelength of 450 nm and at a frequency of 1 application every day (series of photographs to the right).

Hence, the method for antifungal treatment of a turf according to the invention comprises a step consisting in applying blue light on said turf. Preferably, the present invention relates to a method for treating a turf as defined before, having the following technical features, considered alone or in combination:
the wavelength(s) of the blue light is (are) comprised between 390 and 490 nm, more preferably between 400 and 480 nm. Quite preferably, the wavelength of the blue light is 405±5 nm, 415±5 nm, 445±5 nm, 450±5 nm and/or 470±5 nm;
the blue light is applied so that the luminous flux per unit area of the turf is comprised between 20 and 150 W/cm². Quite preferably, said luminous flux is comprised between 40 and 130 mW/cm², between 60 and 110 mW/cm², between 70 and 100 mW/cm²;
the blue light is applied for a time period comprised between 0.3 seconds and 1 hour and 29 minutes. Quite preferably, said duration is comprised between 0.5 seconds and 1 hour and 15 minutes, between 1 second and 1 hour, between 2 seconds and 45 minutes, between 3 seconds and 30 minutes. Advantageously, said duration is comprised between 0.3 and 30 seconds, between 1 and 20 seconds, between 2 and 10 seconds, between 3 and 5 seconds;
the blue light is applied during the day and/or during the night;
the blue light is applied at a frequency of 1 to 60 application(s) every month, 1 to 50 application(s) every month, 1 to 40 application(s) every month, 1 to 30 application(s) every month;
the blue light is applied over one or several day(s), consecutive or not;
the blue light is applied over a time period which could range up to 4 months or more, up to 6 months or more or 12 months or more. Quite preferably, the blue light is applied as long as the deleterious effects of the micromycete(s) are observed on the turf (discoloration, circular spots, etc.);
the blue light is applied at a frequency of 1 passage every day for 30 days; after 4 days of treatment, the pass frequency is reduced to 1 pass every 3 days, for 10 days; afterwards, the frequency is reduced to 1 pass every 5 days for 10 days, then 1 pass every 7 days for 14 days and finally, 1 pass every 15 days as long as necessary throughout the year (summer and/or winter), that is to say as long as the presence of at least one fungal infection by micromycetes is detected and/or in prevention of a resumption of the development and/or of the activity of the micromycetes present in the turf; and/or
the blue light is applied by means of light source(s), selected from among the group constituted by light-emitting diode(s) (LED) and organic light-emitting diode(s) (OLED), More preferably, the light source(s) is (are) arranged on a support tool, automated or not, preferably a robot, so as to facilitate the antifungal treatment and, advantageously, to automate the antifungal treatment. As example, mention may be made of the Turflynx F315 robot whose travel speed amounts to 0.3 m/sec.

The method according to the present invention can be used to treat any fungal disease of turf.

Preferably, the present invention relates to a method as described before for the treatment of fungal diseases selected from among “cold season” fungal diseases and “hot season” fungal diseases, such as:
fusarium wilt (cold or hot), caused by the fungi of the genus Fusarium spp. or Microdochium spp. such as Fusarium nivale, Microdochium nivale and Microdochium majus;
rhizoctonia or brown spots, caused by the fungi of the genus Rhizoctonia, including R. solani or R. cerealis;
“dollar spot”, caused by the fungi of the genus Clarireedia, including C. homoeocarpa, C. monteithiana, C. jacksonii or C. bennettii, or the fungus Sclerotinia homeocarpa;
rusts, for example the crown rust, caused by the fungi (basidiomycetes) of the genera Puccinia spp. and Uromyces spp., for example, P. brachypodii, P. poarum or P. coronata or by fungi by Scleroderma spp., or Hygrophorus spp.;
helminthosporiosis, caused by Drechslera spp. and Bipolaris spp.;
snow rot, caused by Typhula incarnata;
combs of agrostises, caused by Gaeumannomyces graminis;
pyriculariasis, caused by the fungus Magnaporthe oryzae, whose anamorphic form is Pyricularia oryzae;
anthracnosis, caused by Colletotrichum graminicola;
the red thread disease, caused by Laetisaria fuciformis;
summer rot, caused by Sclerotium rolfsii;
Pythium or seedling loss, caused by Pythium spp.;
powdery mildew, caused by Erisyphe graminis;
plasmodia, caused by Mucilago crustacea, Didymium squamulosum, Physarum spp. or Fuligo spp.
fairy rings caused by Marasmius oreade, Trechispora alnicola, T. farinacea, fungi of the genera Melanotus spp. or Cristella spp. like C. confinus.

The present invention is illustrated in a non-limiting manner by the following examples.

### Example 1: In Situ Assessment of the Effect of the Treatment by Application of Blue Light on Turf Infected with Fungal Diseases

### 1. Device for the Treatment of Fungal Diseases of Turf

LED boards have been soldered onto a copper circuit, then encapsulated in an aluminum profile, so as to form a light therapy module. Four of these modules have been grouped together, assembled and transposed onto a Turflynx F315 multifunction robot (Turflynx Ltd, Portugal). In detail, it consists of a set comprising 4×2 modules located at the front of the robot, each of the modules comprising 24 LEDs (namely 24×4×2 LEDs located at the front of the robot) and 1×4 modules located at the rear of the robot, each of the modules comprising 24 LEDs (namely 24×1×4 LEDs located at the rear of the robot) so as to cover the entire surface to be treated. In other words, the Turflynx F315 multifunction robot is provided with a total of 864 Osslon SSL LEDs, emitting a blue light having a 450 nm wavelength, whose absorbed power amounts to 1.74 W and whose rendered power amounts to about 0.781 W. It arises from the foregoing that the rendered total power is about 675 W for a total absorbed power of about 1,500 W. The emission height of the blue light with respect to the ground is 45 mm.

### 2. Phototherapy Protocol

Turf is a sports and leisure turf present in its artificial medium, in particular a golf course.

Microscopic fungi (or micromycetes) are naturally present in the soils, and could in particular be supplied “artificially” by different vectors like wind, shoe soles or machines used for maintenance.

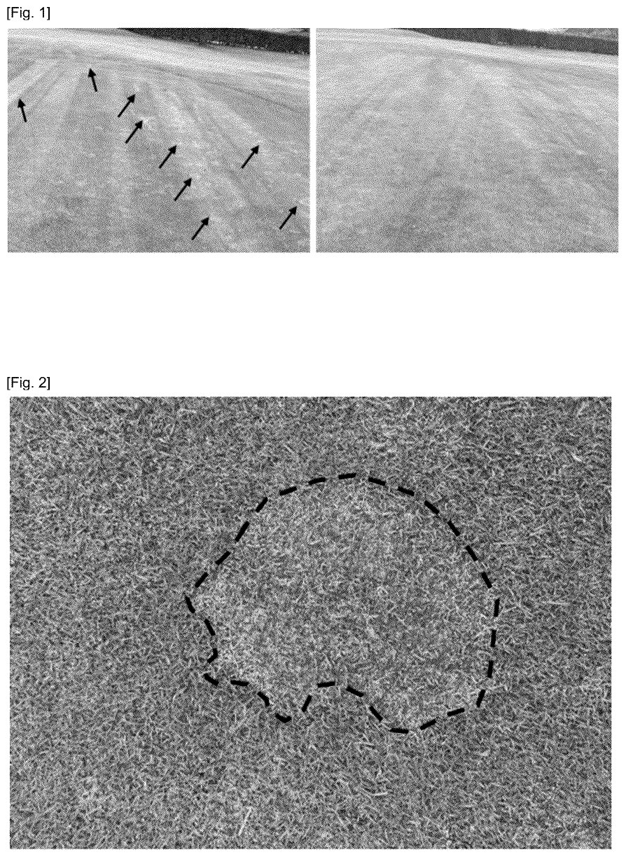

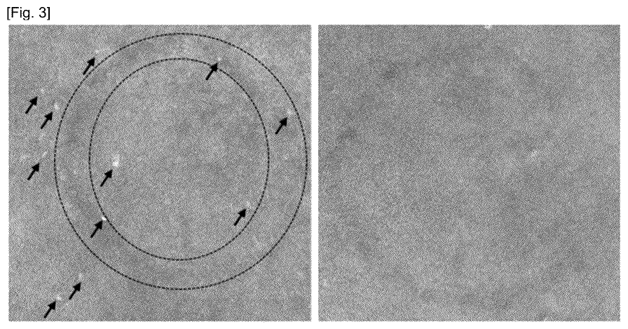

The treatment consists in applying blue light at a wavelength of 450 nm using LEDs, the minimum light power of which amounts de 20 mW/cm² a day, according to the characteristics set out at point 1
at a rate of 21 applications for 30 days with a frequency of 2 applications every day for 4 days, then 1 application every 2 days, for the treatment of cold fusarium wilt (FIG. 1). Each of the applications lasting 1 to 3 seconds; and
at a rate of 10 applications for 30 days with a frequency of 1 application every 3 days for the treatment of cold fusarium wilt and of fairy rings (FIG. 3). Each of the applications having a duration of 1 to 3 seconds.

### 3. Results

a. Treatment of the Turf Suffering from Cold Fusarium Wilt (F. nivale, M. Nivale and M. Majus)

The photograph to the right of FIG. 1 illustrates the turf infected with cold fusarium wilt before treatment by application of the blue light as described in item 2. It appears that the turf has circular spots measuring between 2 and 40 cm in diameter (arrows) and a modification of the color of the turf that appears brown, yellow or white (presence of cotton filaments) at some locations (arrows).

Following the treatment by application of blue light according to the invention (the photograph to the left in FIG. 1; FIG. 2), and 30 days after stoppage of this treatment, the same turf area no longer has circular spots, the grasses have regrown so as to cover the entire area of the golf course and the turf has recovered its natural color (green).

It arises from the foregoing that the mere application of blue light according to the invention on the turf suffering from cold fusarium wilt is effective in reducing the presence and the activity of the micromycetes at the origin of this phytopathology and allows restoring the turf to a natural state enabling the use of the golf course.

b. Treatment of the Turf Suffering from Cold Fusarium Wilt (M. nivale) and from Fairy Rings (Marasmius oreade)

The photograph to the right of FIG. 3 illustrates the turf before treatment by application of the blue light as described in item 2. It appears that the turf features a modification of its color since it appears brown or yellow and has white cotton filaments (arrows) at some locations. Furthermore, the presence of a circle of “green” turf or annular mycelium (the area identified between the dotted lines), the central part of which contains turf, is characteristic of fairy rings. Indeed, the circle of turf so-called “luxuriant” corresponds to the growing mycelium which extracts nutrients from the area located at the center of the circle before growing centrifugally when the resources of the soil are exhausted.

After treatment by application of blue light according to the invention (photograph to the left of FIG. 3), and 30 days after stoppage of this treatment, the same area of the turf no longer has circular spots, the grasses have regrown so as to cover the entire area of the golf course, including the corresponding area inside the annular mycelium, and the turf has recovered its natural color (green).

It arises from the foregoing that the only application of blue light according to the invention on the turf suffering from cold fusarium wilt and fairy rings is effective in reducing the presence and the activity of the micromycetes at the origin of these phytopathologies and allows restoring the turf to a natural state enabling the use of the golf course.

### Example 2: In Vitro Assessment of the Direct Effect of the Treatment by Application of Blue Light on the Growth of the Microdochium nivale Mycelium in Culture

### 1. Equipment and Methods

The M. nivale mycelium is derived from a sample in a golf course and then from a selection intended to confirm the strain of the micromycete and of a culture of the micromycete on a PDA medium (potato/dextrose/agar or dextrorized agar of potato). A 5 mm diameter mycelial plug has been cut in the mycelium of the fungus previously cultured and then this plug has been placed at the center of a Petri dish on a PDA medium and then incubated in the dark at a temperature of 20° C.

A Petri dish in culture has received no treatment and corresponds to the control condition. A Petri dish in culture has been subjected to the application of blue light at a wavelength of 450 nm at a rate of 1 application every day for 6 days, each application lasting 30 seconds. The experiment is carried out in triplicate.

Photographs have been carried out 2, 3, 4, 6 and 12 days after culturing (and correspond to the designation D+2, D+3, D+4, D+6 and D+12, respectively).

### 2. Results

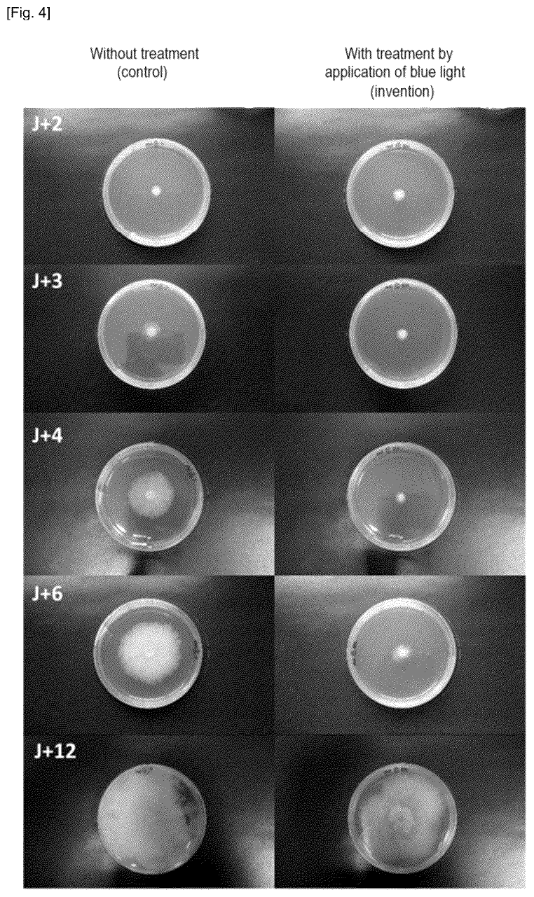

The results are represented by FIG. 4 and show that the absence of treatment leads to a regular growth of the M. nivale mycelium which invades the entire Petri dish at D+12.

The treatment of this micrometre by application of blue light according to the invention induces a control of the growth of the M. nivale mycelium between D+2 and D+6. The photography at D+12 under the treated condition shows that the growth of this mycelium is not inhibited but is controlled/maintained since the fungus develops but at a level lower than what is observed in the control condition.

Furthermore, it should be noted that after stoppage of the treatment by application of blue light, namely after 12 days of treatment, the mycelium resumes a development equivalent to the untreated condition.

It arises from the foregoing that the mere application of blue light according to the invention allows controlling/maintaining the growth of the M. nivale mycelium cultivated in vitro. In particular, these data demonstrate that the application of blue light according to the invention produces a direct effect on the growth and/or the activity of a micromycete.

1. A method for the antifungal treatment of a turf, comprising a step consisting in applying blue light on said turf.

2. The method according to claim 1, wherein the wavelength(s) of the blue light is (are) comprised between 380 and 500 nm.

3. The method according to claim 1, wherein the blue light is applied so that the luminous flux per unit area of the turf is comprised between 20 and 150 W/cm².

4. The method according to claim 1, wherein the blue light is applied for a tile period comprised between 0.3 seconds and 1 hour 29 minutes.

5. The method according to claim 1, wherein the blue light is applied during the day and/or during the night.

6. The method according to claim 1, wherein the blue light is applied at a frequency of 1 to 60 application(s) per month, over one or several days, consecutive or not, over a period which could range up to 12 months or more.

7. The method according to claim 1, wherein the blue light is applied by means of light source(s), selected from the group constituted by light-emitting diode(s) (LED) and organic light-emitting diode(s) (OLED).

8. The method according to claim 7, wherein the light source(s) is (are) arranged on a support tool, automated or not.

9. The method according to claim 1, wherein the fungi are selected from the group consisting of Fusarium spp. preferably Fusarium nivale; spp.; Microdochium spp., preferably Microdochium nivale and Microdochium majus; spp.; Rhizoctonia solani; Rhizoctonia cerealis; Clarireedia homoeocarpa; Clarireedia monteithiana; Clarireedia jacksonii; Clarireedia bennettii; Sclerotinia homeocarpa; Puccinia spp., preferably Puccinia brachypodii, Puccinia poarum and Puccinia coronata; spp.; Marasmius oreads; Scleroderma spp.; Hygrophorus spp.; Uromyces spp.; Drechslera spp.; Bipolaris spp.; Typhula incarnata; Gaeumannomyces graminis; Magnaporthe oryzae; Pyricularia oryzae; Colletotrichum graminicola; Laetisaria fuciformis; Sclerotium rolfsii; Phytium spp.; Erisyphe graminis; Mucilago crustacea; Dineodymium squamulosum; Physarum spp.; Fuligo spp.; Marasmius oreade; Trechispora alnicola; Trechispora farinacea; Melanotus spp. Cristella spp.; and mixtures thereof.

10. The method according to claim 1, wherein the turf comprises grasses, selected from the group consisting of:
agrostis;
meadow-grass;
fescue; and/or
English ryegrass.
